(19) United States
(12) Patent Application Publication
(10) Pub. No.: US 2007/0269457 A1
(43) Pub. Date: Nov. 22, 2007

(54) Immunotherapeutic compositions and methods

(75) Inventors: Kayvan R. Niazi, Novato, CA (US); Shahrooz Rabizadeh, San Rafael, CA (US); Dale E. Bredesen, Novato, CA (US)

Correspondence Address: MEDLEN & CARROLL, LLP, 101 HOWARD STREET, SUITE 350, SAN FRANCISCO, CA, 94105, US

(73) Assignee: The Buck Institute For Age Research

(21) Appl. No.: 11/435,032

(22) Filed: May 16, 2006

(51) Int. Cl. A61K 39/145; C12N 15/86; C12N 7/01; C07K 14/11

(52) U.S. Cl. 424/209.1; 435/235.1; 435/456; 530/350

(57) ABSTRACT

A vaccine is described which provides protection against a broad spectrum of viral strains, including but not limited to influenza A strains such as H1N1, H3N2, and H5N1. Embodiments of the present invention offer a number of advantages over conventional vaccine, in that they are cheaper to synthesize, more stable, easily scalable, and amenable to further genetic manipulation. Most importantly, certain embodiments of the present invention contemplate features, including but not limited to endosomal targeting, which result in a more robust immune response.

### FIELD OF INVENTION

Embodiments of the present invention are relevant to the fields of immunotherapy and vaccine development. In one embodiment, the invention contemplates novel vaccine preparations and methods. In one embodiment, a strategy is employed so as to provide protection against more than one virus strain. For example, in one embodiment, the present invention contemplates a vaccine which provides protection against a broad spectrum of influenza A strains including but not limited to H1N1, H3N2, and H5N1.

### BACKGROUND

Vaccination provides one means of preventing disease associated with a variety of microbial pathogens including viruses (e.g. poxvirus, poliovirus, measles virus, mumps virus, etc.) and bacteria (Clostridium tetani, Bordetella pertussis, Haemophilus influenzae, etc.). Most vaccine preparations contain either attenuated (or otherwise weakened) organisms or highly purified microbial antigens as observed in subunit vaccines. The induction of an effective antiviral immune response using these live attenuated virus vaccines, however, are known to result in a significant rate of adverse events (i.e., for example, autism). Kennedy et al., “Measles virus infection and vaccination: potential role in chronic illness and associated adverse events” Crit Rev Immunol. 24(2):129-56 (2004).

Immunization experiments with recombinant DNA encoding microbial proteins of various origins have provided demonstrate their potential utility as vaccines. In general, these plasmids contain a strong eukaryotic promoter element which drives the expression of the microbial gene of interest. The resulting protein is usually expressed in the cytoplasm where it is proteolytically degraded into small peptides (or epitopes) and believed to be presented by MHC class I to cytotoxic CD8+ T cells. Although plasmid DNA offers several advantages over the attenuated or subunit vaccine preparations, immunological comparison of first generation plasmid vaccines based on the basic construct arrangement described above to subunit or attenuated vaccines have demonstrated less then favorable results to date both in the magnitude and the duration of the ensuing immune response.

What is needed is an improved vaccine strategy that generates a robust response and provides a broad spectrum of protection.

### SUMMARY OF THE INVENTION

Embodiments of the present invention are relevant to the fields of immunotherapy and vaccine development. In one embodiment, the invention contemplates novel vaccine preparations and methods. In one embodiment, a strategy is employed so as to provide protection against more than one virus strain. In one embodiment, the present invention contemplates a vaccine which provides protection against a broad spectrum of influenza A strains including but not limited to H1N1, H3N2, and H5N1. Embodiments of the present invention offer a number of advantages over conventional vaccine, in that they are cheaper to synthesize, more stable, easily scalable, and amenable to further genetic manipulation. Most importantly, certain embodiments of the present invention contemplate features, including but not limited to endosomal targeting, which result in a more robust immune response.

One important component of the improved vaccine strategy of the present invention is the selection of the antigen or “immunogen.” While natural immunogens (such as native proteins) are contemplated, it is preferred, because of heterogeneity among strains due to the natural genetic drift driven by mutations, that designed (i.e. “modified” or “non-native”) immunogens be employed. In one embodiment, the present invention contemplates a method of designing the immunogen, comprising a comparison made among the protein sequences of at least two strains of a virus (or, alternatively, at least two viruses) by sequence homology so as to design a cDNA encoding a sequence with a high degree of identity (greater than 80%, more preferably greater than 90%, still more preferably greater than 95% identity at the amino acid level) with natural variants. It is not intended that the present invention be limited to the particular protein sequence used in this comparison. In one embodiment, the comparison is made with viral matrix proteins. In another embodiment, the comparison is made with viral core proteins.

When matrix proteins are used, it is not intended that the present invention be limited to matrix proteins of only one viral type. Indeed, a variety of viruses are contemplated. For example, the present invention contemplates using native or optimized matrix protein sequences from M proteins of a number of different viruses including but not limited to: i) Ebola virus (Jasenosky et al., “Filovirus budding” Virus Res. 106:1B1-8 (2004); Jasenosky et al., “Ebola virus VP40-induced particle formation and association with the lipid bilayer” J. Virol. 75:5205-14 (2001); and Timmins et al., “Vesicular release of Ebola virus matrix protein VP40” Virology 283: 1-6 (2001)); ii) vesicular stomatitis virus (Jayakar et al., “Rhabdovirus assembly and budding” Virus Res. 106:117-32 (2004); Li et al., “Viral liposomes released from insect cells infected with recombinant baculovirus expressing the matrix protein of vesicular stomatitis virus” J. Virol. 67:4415-20 (1993); and Sakaguchi et al., “Double-layered membrane vesicles released from mammalian cells infected with Sendai virus expressing the matrix protein of vesicular stomatitis virus” Virology 263:230-43 (1999)), iii) HIV, iv) EBV and, v) influenza virus (Gomez-Puertas et al., “Influenza virus matrix protein is the major driving force in virus budding” J. Virol.74:11538-47 (2000)). Additional viruses include but are not limited to Influenza B, HIV, parainfluenza, rabies virus (rhabidovirus), mumps virus, measles virus, dengue virus, Newcastle virus, and morbillivirus.

Moreover, the approach described herein is not limited to using only matrix proteins or modified matrix proteins. A variety of conserved antigens (be they M-like, core proteins, polymerases, etc.) can be delivered using vectors encoding such a conserved antigen (or modified antigen with high homology to a variety of strains of the particular virus) as a fusion protein with endosomal targeting sequences and/or secretory sequences. In particular, the present invention contemplates embodiments utilizing native and non-native proteins such as papillomavirus E6, HIV polymerase, and hepatitis C virus core protein.

Furthermore, the entire protein need not be encoded by nucleic acid of the vector. In one embodiment, the conserved region of a viral protein is used (either the native sequence or a non-native sequence designed by comparing variations among strains (or related viruses). Again, it is not intended that the present invention be limited to the particular virus, protein, or conserved region. For example, a native, conserved region of an HIV protein may be used or a non-native sequence can be designed based on a comparison between HIV strains such that immunization results in protection against variants. Where a conserved region (native or non-native) is employed, the present invention contemplates fusion proteins (and constructs encoding such fusion proteins) comprising endosomal targeting sequences and/or secretory sequences.

With regard to influenza virus, comparisons of the protein sequences of an assortment of influenza A virus Matrix proteins by sequence homology lead to the design of a cDNA encoding a Matrix sequence with high degrees of identity (greater than 96% amino acid identity) with a significant number of published matrix sequences from H1N1, H3H2, H5N1, as well as other influenza A viruses. This is a preferred immunogen of the present invention.

A second important component of the improved vaccine strategy is the use of targeting sequences and/or secretory sequences. Preferred targeting sequences are endosomal targeting sequences, such as LAMP-1 and members of the CD1 family. Alternatively, LIMP-2 sequences, invariant chain sequences, and MHC II sequences can be used to traffic antigens into the endosomes. In one embodiment, the present invention contemplates DNA constructs comprising nucleic acid sequences encoding an optimized (non-native) matrix protein and endosomal targeting sequences (e.g. as a fusion protein). On the other hand, leader sequences or secretory sequences may be employed. It is not intended that the present invention be limited to particular leader sequences or secretory sequences. In one embodiment, the present invention contemplates DNA constructs comprising nucleic acid sequences encoding an optimized (non-native) matrix protein and one or more secretory sequences (such as the tPA sequence: MDAMKRGLCCVLLLCGAVFVSPS, SEQ ID NO:).

Additional components may (optionally) be added or used in conjunction with the immunogens of the present invention, such as adjuvants and other immune activators. In some embodiments, adjuvants comprising liposomes are contemplated, including but not limited to Vaxfectin (Vival, Inc.), DMPC/cholesterol, and DC/cholesterol. In another embodiment, immunoadjuvants (or DNAs encoding immunoadjuvants) such as IL-2, IL-12, IL-15, GM-CSF, B7.1, and the like are contemplated. More traditional adjuvants such as incomplete Freund's adjuvant or mineral salts such as alum or calcium phosphate are contemplated as well. Alternatively, emulsions containing MF59, QS21, squalene-containing preparations and montanide may be employed. Alternatively, natural or synthetic bacterial products comprising monophosphoryl lipid A (MPL), RC-529 (synthetic MPL), holotoxins like CT, PT and LT, or CpG oligonucleotides may be employed in conjunction with the immunogens of the present invention. In still further embodiments, virosomes like ISCOMS (a structured complex of saponins and lipids) may be employed.

In one embodiment, such activators or adjuvants may be employed in particle form, such as nanoparticles or microspheres. The particles may be made of a variety of substances, including polymers (e.g. PLGA). The activators or adjuvants can be on the particles, in the particles, or even the particles themselves. In one embodiment, an activator (e.g. thapsigargin) is conjugated to liposomes, microbubbles, nanoparticles or biomatter (protein, lipid, carbohydrate or nucleic acid).

The present invention contemplates methods and compositions. With respect to compositions, the present invention, in one embodiment, contemplates vectors comprising DNA sequences encoding one or more fusion proteins, said fusion protein comprising a native or a non-native viral matrix protein (e.g. a non-native Influenza A matrix protein described herein) and one or more endosomal targeting sequences (e.g. LAMP-1 or CD1 isoform). In one embodiment, the present invention contemplates vectors comprising DNA sequences encoding a fusion protein comprising a native or non-native viral matrix protein and one or more secretory sequences. In one embodiment, the present invention contemplates vectors comprising DNA sequences encoding a fusion protein comprising a native or non-native viral matrix protein, and both a secretory sequence and an endosomal targeting sequence. In one embodiment, the vectors comprise one or more internal ribosomal entry sites (IRES).

In one embodiment, the present invention contemplates vaccines comprising one or more vectors comprising DNA sequences encoding the above described fusion proteins. Alternatively, vaccines are contemplated comprising a first vector comprising DNA sequences encoding a first native conserved viral protein (e.g. a native matrix protein from a first strain of Influenza A) and a second vector comprising DNA sequences encoding a second native conserved viral protein (e.g. a native matrix protein from a second strain of Influenza A). In one embodiment, the first and second native conserved viral proteins are encoded on the same vector. In these various embodiments, it is preferred that the proteins be expressed as fusion proteins (e.g. with endosomal targeting sequences, secretory sequences or both). In one embodiment, the fusion proteins comprise a CD1 isoform-specific cytoplasmic tail endosomal targeting motif.

The present invention also contemplates vectors comprising additional immune target antigens and an IRES sequence to drive a differently targeted version of the same protein to try to optimally deliver it to both classes of T cells. In one embodiment, the vector comprises DNA encoding a CD1c, LAMP, or targeted version of an antigen followed by an IRES sequence and cytoplasmic version of the same antigen to try to overload both the MHC II and I pathways.

The present invention also contemplates the resulting fusion proteins (whether purified or unpurified) as compositions. In one embodiment, the present invention contemplates vaccines wherein the fusion protein (e.g. without the vector) is administered.

As mentioned above, methods are also contemplated, including methods where one or more of the above-described vectors (or fusion proteins) are administered to a cell, or a host (including a human). In one embodiment, the present invention contemplates a method, comprising; a) providing, a vector comprising DNA sequences encoding a fusion protein comprising a non-native viral matrix protein and one or more endosomal targeting sequences; and a cell capable of being transfected by said vector (e.g. a cell culture grown ex vivo); b) transfecting said cell with said vector so as to create a transfected cell under conditions such that said fusion protein is expressed. In one embodiment, the method further comprises c) introducing said transfected cell to a host. In one embodiment, said endosomal targeting sequence is a member of the CD1 family. In one embodiment, said endosomal targeting sequence is a LAMP-1 sequence. In one embodiment, the present invention contemplates a method, comprising; a) providing, a vector comprising DNA sequences encoding a fusion protein comprising a non-native viral matrix protein and one or more endosomal targeting sequences; and a host; b) administering said vector to said host (thereby transfecting a portion of said host's cells with said vector) under conditions such that said fusion protein is expressed.

It is not intended that the present invention be limited by the route of administration (e.g. oral, intravenous, subcutaneously, transdermally, intramuscularly, etc. can be employed). In one embodiment, the vector is administered intradermally. In another embodiment, the administration is by the intranasal route. In one embodiment, cationic liposomes (e.g. T9-encapsulated cationic liposomes) are contemplated for delivery the DNA to the immune system by the intranasal route. In another embodiment, the vector(s) are delivered in an aerosol or mist for inhalation. In a preferred embodiment, the DNA constructs are administered as a needle-free vaccine that is inhaled.

In one embodiment, said endosomal targeting sequence is a member of the CD1 family. In one embodiment, said endosomal targeting sequence is a LAMP-1 sequence. In one embodiment, said host is a bird, whether wild or domesticated (e.g. a chicken). In a preferred embodiment, said host is a human. In one embodiment, said fusion protein further comprises one or more secretory sequences. In one embodiment, said fusion protein further comprises a transmembrane domain. In one embodiment, fusion protein further comprises a leader sequence.

In one embodiment, the present invention contemplates a method, comprising; a) providing, a vector comprising DNA sequences encoding a fusion protein comprising a non-native viral matrix protein and one or more secretory sequences; and a host; b) transfecting said host with said vector under conditions such that said fusion protein is expressed.

Definitions

The term “matrix protein”, or “M protein” as used herein, means any viral protein localized between the envelope and the nucleocapsid core. The matrix protein of many enveloped RNA viruses are believed to play a role in virus assembly and budding. Freed, E. O., “The HIV-TSGI01 interface: recent advances in a budding field” Trends Microbiol. 11:56-9 (2003); Jasenosky et al., “Filovirus budding” Virus Res. 106:1B1-8 (2004); Jayakar et al., “Rhabdovirus assembly and budding” Virus Res. 106:117-32 (2004); Peeples M. E., “Paramyxovirus M proteins: pulling it all together and taking it on the road” pp. 427-456. In: The Paramyxoviruses, Ed: D. W. Kingsbury, Plenum, New York, N.Y. (1991); Pornillos et al., “Mechanisms of enveloped RNA virus budding” Trends Cell Biol. 12:569-79 (2002); Schmitt et al., “Escaping from the cell: assembly and budding of negative-strand RNA viruses” Cuff Top Microbiol Immunol 283:145-96 (2004); and Takimoto et al., “Molecular mechanism of paramyxovirus budding” Virus Res. 106:133-45 (2004).

A “native” matrix protein is naturally occurring (e.g. wild-type). A non-native matrix protein is one that does not naturally exist. An “optimized” matrix protein is a non-native matrix protein which has been modified in sequence so as to take into account variations among strains. When designing modified or optimized matrix proteins, a high degree of identity (greater than 80%, more preferably greater than 90%, still more preferably greater than 95% identity at the amino acid level) with natural variants is preferred.

The term “vector” as used herein, refers to any nucleotide sequence comprising one or more exogenous operative genes capable of expression within a cell. For example, in certain embodiments, a vector of the present invention may comprise a nucleic acid encoding a viral matrix protein (whether native or non-native).

The term “transfect” or “transfecting” as used herein, refers to any mechanism by which a vector may be incorporated into a cell, including but not limited to a host cell. A successful transfection results in the capability of the host cell to express any operative genes carried by the vector. Transfections may be stable or transient. One example of a transient transfection comprises vector expression within a cell, wherein the vector is not integrated within the host cell genome. Alternatively, a stable transfection comprises vector expression within a cell, wherein the vector is integrated within the host cell genome.

The term “host” as used herein, refers to any organism capable of being transfected. It is not intended that the present invention be limited by the nature of the host. A host may be an avian host (i.e., for example, a chicken) or a mammalian host (i.e., for example, human, mouse, dog, rat, cow, sheep, etc.).

### BRIEF DESCRIPTION OF THE FIGURES

FIG. 1 presents one embodiment of the nucleic acid sequence (SEQ ID NO: 1) and amino acid sequence (SEQ ID NO:2) of the pVAX-M construct.

FIG. 2 presents one embodiment of the nucleic acid sequence (SEQ ID NO: 3) and amino acid sequence (SEQ ID NO:4) of the pVAX-M-CD1a construct.

FIG. 3 presents one embodiment of the nucleic acid sequence (SEQ ID NO: 5) and amino acid sequence (SEQ ID NO:6) of the pVAX-M-CD1b construct.

FIG. 4 presents one embodiment of the nucleic acid sequence (SEQ ID NO: 7) and amino acid sequence (SEQ ID NO:8) of the pVAX-M-CD1c construct.

FIG. 5 presents one embodiment of the nucleic acid sequence (SEQ ID NO: 9) and amino acid sequence (SEQ ID NO:10) of the pVAX-M-CD1d construct.

FIG. 6 presents several embodiments demonstrating the basic structure of pVAX-M constructs. a) one embodiment of a CD1b construct: L: leader sequence. TM: a transmembrane domain, wherein at least a portion is extracellular. CT: cytoplasmic tail. b) one embodiment of a pVAX-M construct. M: matrix protein. Arrow: cytomegalovirus promoter. c) a representative panel of pVAX-M-CD1 constructs (i.e., CD1 a-d). L: leader sequence. M: matrix protein. TM: a transmembrane domain, wherein at least a portion is extracellular.

FIG. 7 presents one embodiment of the nucleic acid sequence (SEQ ID NO: 11) and amino acid sequence (SEQ ID NO: 12) of the pIRES-Puro3-M-Flag construct.

FIG. 8 presents one embodiment of the nucleic acid sequence (SEQ ID NO: 13) and amino acid sequence (SEQ ID NO:14) of the pVAX-M-LAMP1 construct.

FIG. 9 presents one embodiment of the nucleic acid sequence (SEQ ID NO: 15) and amino acid sequence (SEQ ID NO:16) of the pVAX-Sig-M construct. The Sig-M construct is essentially a deletion construct of the M-CD1 or M-LAMP constructs where the transmembrane and cytoplasmic tails have been removed and replaced with a stop codon. As a result, the remaining sequence is the CD1b leader sequence and the M protein.

FIG. 10 presents exemplary data showing the survival of mice immunized with various M-CD1 plasmids following a challenge with P815 mastocytoma cells modified to express M protein. Open Diamond: Empty Vector. Solid Diamond: M protein Vector. Open Triangle. M-CD1a Vector. Solid Triangle: M-CD1b Vector. Open Circle: M-CD1c Vector. Solid Circle: M-CD1d.

FIG. 11 presents exemplary data showing the time course of tumor development in mice following the injection of various concentrations of P815 mastocytomas cells. Solid Diamonds: 10,000 P815 cells. Open Square: 50,000 P815 cells. Solid Circles: 250,000 P815 cells.

FIG. 12 shows the map for the commercially available pVAX1 expression plasmid.

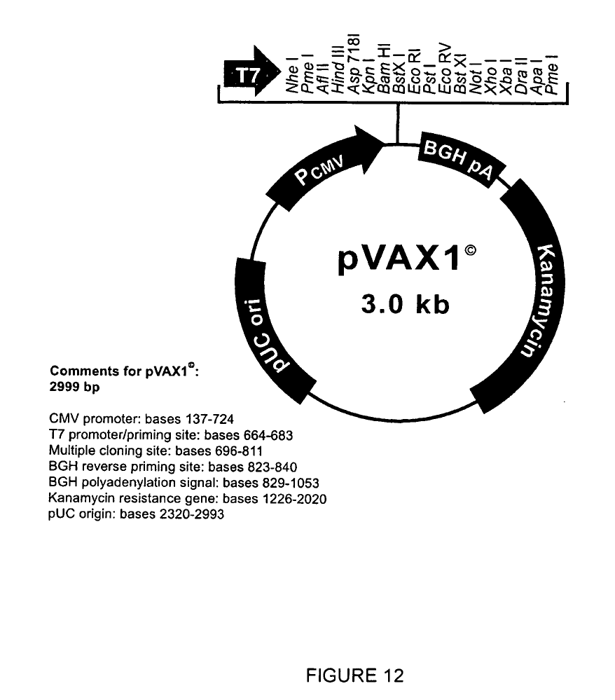

### DETAILED DESCRIPTION OF PREFERRED EMBODIMENTS

Embodiments of the present invention are relevant to the fields of immunotherapy and vaccine development. In one embodiment, the invention contemplates novel vaccine preparations and methods. In one embodiment, a strategy is employed so as to provide protection against more than one virus strain. Embodiments of the present invention offer a number of advantages over conventional vaccine, in that they are cheaper to synthesize, more stable, easily scalable, and amenable to further genetic manipulation. Most importantly, certain embodiments of the present invention contemplate features, including but not limited to endosomal targeting, which result in a more robust immune response.

In one embodiment, the present invention contemplates a vaccine which provides protection against a broad spectrum of influenza A strains including but not limited to H1N1, H3N2, and H5N1. In one embodiment, the vaccine is a recombinant vaccine comprising nucleic acid (e.g. cDNA) encoding an Influenza A matrix protein (native or non-native) that is targeted to one or more intracellular locations through the addition of one or more endosomal targeting sequences. A preferred endosomal targeting sequence is derived from the human CD proteins (i.e., for example, CD1(a-e).

In some embodiments, non-native matrix proteins are preferred. For example, in one embodiment, the cDNA was designed to encode a non-native matrix protein that is highly homologous to the matrix proteins of a broad spectrum of Influenza A strains including, but not limited to, H1N1, H3N2, and H5N1. Immunizations performed using this embodiment of the invention provided protection against challenge with a genetically-engineered cancer cell line expressing the matrix protein indicating the potential utility of this platform in providing broad protection against a variety of Influenza A viral strains in animals and people.

While it is not necessary that one understand precise mechanisms in order to construct the vectors or carry out the methods of the invention, it is believed that, CD4+ helper T lymphocytes have demonstrated an important role in modulating the immunological outcome of vaccination. Unlike CD8+ T cells which recognize cytoplasmic protein fragments in the context of MHC class I, CD4+ T cells recognize proteolytic antigen fragments of proteins present in various intracellular endosomal compartments in the context of the MHC class II molecule. Vaccine antigens gain access to endosomes following internalization from the extracellular fluid. Regardless, once appropriately activated, it is believed that CD4+ T cells release cytokines which determine whether antibody-secreting B cells or cytotoxic CD8+ T cells would best serve to contain the infection and are thus typically classified as “helper” cells.

Perhaps of greatest significance to vaccine design, it is believed that CD4+ T cells play an integral role in the generation of memory CD8+ T cells and the expeditious response to previously encountered intracellular pathogens such as viruses and certain families of bacteria. The importance of CD4+ T cells to the generation of immunological memory is further illustrated in studies using plasmid DNA where the antigen of interest is fused to targeting sequences derived from endosomal resident proteins (leading to its traffic into the MHC class II antigen presentation pathway). For instance, whereas mice immunized with first generation plasmid DNA encoding cytoplasm-localized antigen demonstrate a sub-optimal immune response against antigen, mice immunized with DNA constructs encoding antigen fused to the lysosome-associated membrane protein (LAMP-1) endosomal targeting sequence demonstrate significantly increased frequencies of both antigen specific CD4+ and CD8+ T cells as well as protection against previously established cancer. Ji et al., “Targeting human papillomavirus type 16 E7 to the endosomal/lysosomal compartment enhances the antitumor immunity of DNA vaccines against murine human papillomavirus type 16 E7-expressing tumors” Human Gene Ther. 10:2727-2740 (1999). Fusion of the LAMP-1 targeting sequence to antigen directs its traffic to a subset of endosomes which includes late endosomes and lysosomes/MHC class II compartments (or MIIC). Distinct types of endosomes contain different pools of proteases and pH conditions, such that optimal antigen processing and presentation by a particular MHC class II protein for any given antigenic epitope may occur in a very restricted number of endosomal compartments. Given the polymorphic nature of MHC class I and II in higher mammals, a construct that elicits an immune response in one group of recipients may not do so all individuals. However, it is not necessary that the constructs of the present invention generate an immune response in all recipients. While broad applicability is desired, vaccines have utility even where there are non-responders (or people with adverse responses) in the population. Moreover, embodiments of the present invention permit immunization with panels of constructs (e.g. two or more vectors) which broadly target various intracellular locations. Such strategies enhance the ability to provide protection to a large population.

The human CD1family of antigen presentation molecules is comprised of five isoforms (a-e), four of which (a-d) are unique in their ability to present non-peptide antigens to T cells. Like LAMP-1, the CD1 proteins traffic within the endosomal system. Moody et al., “Intracellular pathways of CD1 antigen presentation” Nat. Rev. Immunol. 3:11-22 (2003). However, unlike LAMP-1, which resides mostly in late endosomes/lysosomes/MIIC, each CD isoform displays a unique trafficking pattern to distinct subsets of endosomal compartments, directed by a tyrosine-based targeting motif located in the cytoplasmic tail. CD1a accumulates predominantly in sorting/recycling endosomes, CD1b traffics similar to LAMP-1 in trafficking to late endosomes/lysosomes/MIIC, CD1c is usually present in sorting, recycling, early, and late endosomes, and CD1d is found throughout the endosomal pathway although it too appears to be most prevalent in the more mature endosomal compartments. Many of the compartments through which CD1 proteins traffic also contain MHC class II and accessory proteins of the MHC class II antigen presentation pathway. These observations led us to ascertain whether CD1-targeted antigens enter the MHC class II presentation pathway in an isoform-specific manner. We found the human melanoma protein MART-1 requires fusion to the CD1b targeting sequence for optimal immune activation in DBA/2 (H-2^{b}) mice while the Mycobacterium tuberculosis antigen ESAT-6 is most immunogenic when targeted by the CD1c targeting motif. Our further in vitro studies demonstrate that a synthetic MHC class II epitope is best presented by fusion to the CD1a tail in HLA-DRB5+ human antigen presenting cells. These data indicate that optimal processing for a given epitope is likely to vary from intracellular compartment to compartment.

Because of these results, we hypothesized that we could utilize the CD1-derived targeting sequences to direct the traffic of the Influenza A virus matrix 1 (M) protein to the appropriate intracellular compartments for optimal presentation. The selection of the matrix protein was due to the fact that it demonstrates better sequence conservation. By contrast, the influenza virus proteins haemaglutinnin (HA) and neuraminidase (N), which are the primary targets of attenuated or subunit vaccines, undergo two different classes of genetic evolution (called genetic drift and genetic shift) from virus generation to generation. This variability makes these vaccines difficult to generate and distribute in sufficient quantity to provide benefit and occasionally ineffective due to the hypervariability of the target antigens.

While the M protein demonstrates significant sequence conservation between different strains of Influenza A and Influenza B viruses, M protein is not exposed to serum in intact viruses and immunization strategies using M protein which generate antibody secreting B cells have not been effective against flu infection. Nonetheless, the present invention contemplates, in one embodiment, using (both native and non-native) M protein as an antigen for immunization strategies focused on engendering cytotoxic T cells.

The use of an M protein vaccine to protect in vivo against an avian flu virus is demonstrated in Example IX. It is expected that these initial results can be improved since the approach has not yet optimized. The CD1-targeted DNA constructs may be further optimized through inclusion of an IRES-M expression construct allowing for the simultaneous traffic of M to both the endosomal pathway to activate CD4+ T cells and the cytoplasm to optimally activate CD8+ T cells. The CD1-targeted M protein encoding plasmids will be further tested in live flu challenge models to confirm their efficacy against influenza virus infections.

That the present approach will demonstrate that DNA-encoded M protein constructs can provide protection against various unrelated strains of flu virus is supported by first generation systems. For example, using a first generation plasmid system, mice can be protected against two different influenza strains. Ozaki et al., “Cross-reactive protection against influenza A virus by a topically applied DNA vaccine encoding M gene with adjuvant” Viral Immunol. 18:373-380 (2005). It is important to note, however, that the immunization protocol utilized required five immunizations as well as the use of additional immunological activators including cholera toxin and CpG DNA which is expensive and not likely to be necessary using the more optimized CD1-targeted M-encoding DNA vaccines described above.

The present invention contemplates that embodiments described herein will provide protection against live infection. Moreover, the constructs can be further modified to express cargo antigens derived from a host of other pathogens including, but not limited to, pox virus, respiratory syncytial virus (RSV), Epstein-Barr virus (EBV), etc. Lastly, due to haplotypic differences in the human population, the derivation of a universal influenza vaccine using the CD1 -based system described above is likely to include a combination of the variously targeted M constructs.

### EXPERIMENTAL

### EXAMPLE I

Creation Of Matrix Protein Plasmid Constructs

This example demonstrates the production of several embodiments of matrix protein plasmid constructs suitable for generating influenza vaccine immunogens.

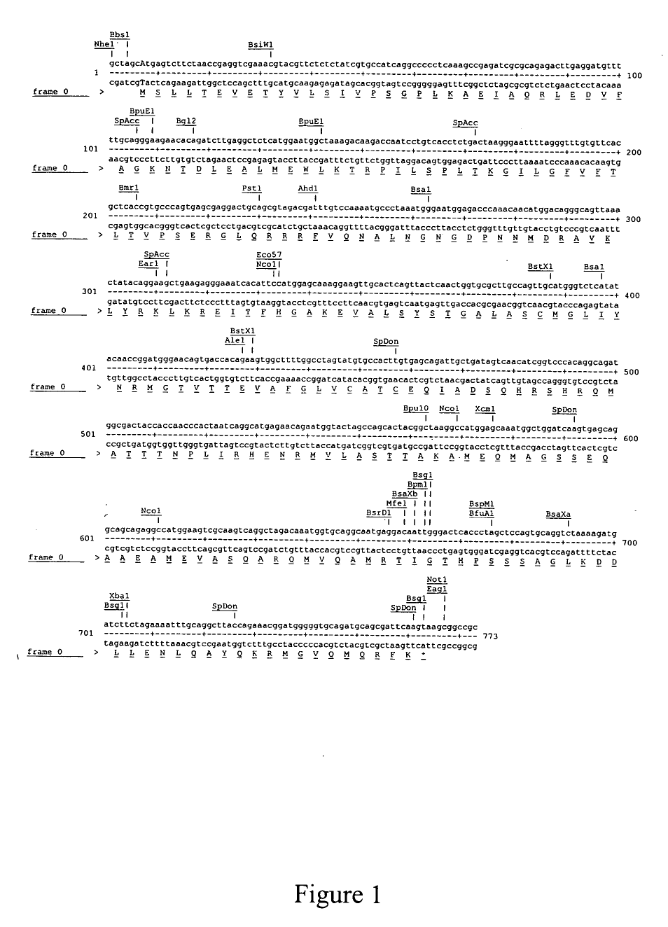

A cDNA encoding a matrix sequence (pVAX-M) was designed using sequence homology techniques by comparing the sequences of an assortment of influenza A virus matrix proteins. pVAX-M was then shown to have a high degree of sequence identity with other known matrix proteins including, but not limited to, H1N1, H3H2, H5N1, and other Influenza A viruses. The nucleotide and amino acid sequence of pVAX-M (with flanking 5′ NheI and 3′NotI sites) are shown in FIG. 1.

The pVAX-M cDNA construct shown in FIG. 1 was generated using an overlapping oligonucleotide technique (Operon, Huntsville, Ala.) and polymerase chain reaction using Pfu DNA polymerase (Stratagene, San Diego, Calif.). The resulting PCR product was digested with NheI and NotI and ligated into NheI/NotI-digested pVAX vector DNA (Invitrogen, Carlsbad, Calif.). Plasmid DNA from the resulting clones was purified and verified by DNA sequencing resulting in the generation of the pVAX-M plasmid.

The pVAX-M plasmid was used as a template for the addition of targeting sequences using additional overlapping oligonucleotides. For example, the leader peptide and transmembrane domains of CD1b were added to the 5′ and 3′ termini, respectively, of the M cDNA using overlapping oligonucleotides. The stop codon encoded within the 3′ end of the original M cDNA was removed to allow translational read-through. The endosomal targeting sequences derived from CD1 isoforms and LAMP-1 were added by PCR using oligonucleotides that encode a novel stop codon at the 3′ end of each construct followed by a NotI site to allow for directional ligation into the pVAX1 construct.

The pVAX-M construct was used as a template for the construction of a cDNA encoding the matrix protein (M protein) whose sequence is described above encoding an additional C terminal Flag epitope tag sequence followed by a stop codon and NotI site to allow directional ligation into a pIRES-Puro2 vector (Clontech, Mountain View, Calif.) resulting in the generation of the pIRES-Puro2-M-Flag DNA construct. Flag epitopes are used to facilitate the isolation and detection of conjugated proteins.

### EXAMPLE II

Mastocytoma Cell Transformation Using pIRES-Puro3-M-Flag

This example presents one embodiment where M protein immunogens may be prepared using transformed cell culture protein expression techniques.

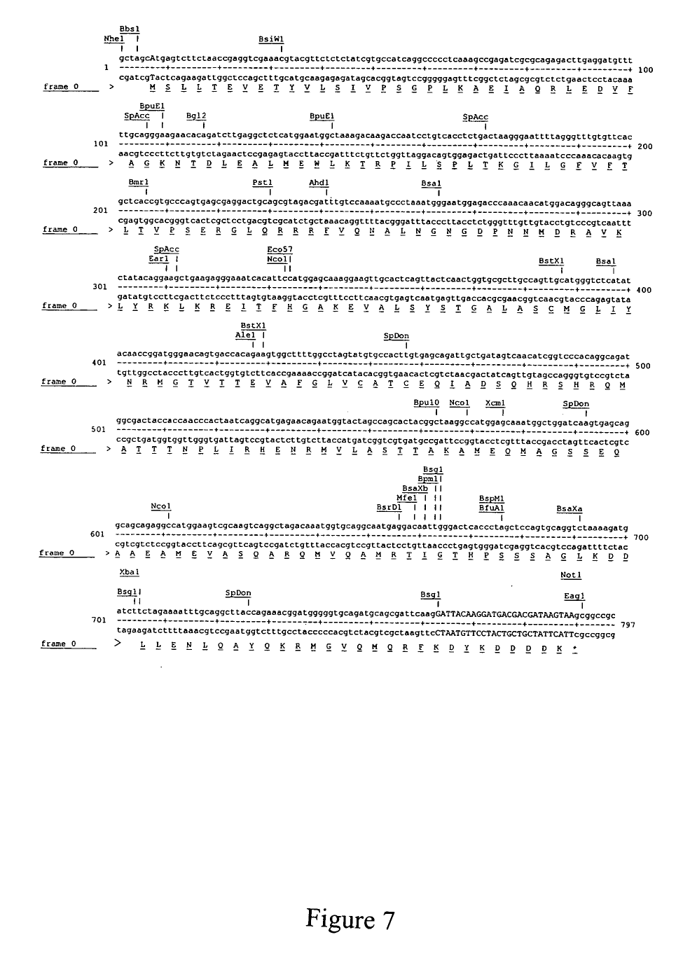

A pIRES-Puro3-M-Flag construct is created according to Example I. FIG. 7. This construct is further purified and then used to transfect a mouse mastocytoma cell line P815 (H-2^{b}) by electroporation using a Biorad GenePulser II electroporator. The P815 mastocytoma cell line is available from American Tissue Type Collection (Manassas, Va.) and can be maintained in DMEM/10%FBS (CellGro, Herndon, Va.) in a humidified 37° C. incubator in the presence of 5% CO₂.

Stable transfectants (P815-M) are selected using 300 ng/ml puromycin and intracellular expression of the M protein is verified by standard intracellular flow cytometry techniques using the anti-Flag M2 antibody (Sigma-Aldrich, St. Louis, Mo.).

### EXAMPLE III

Immunization by Matrix Protein Vaccines

This example describes the immunization of an animal by viral matrix protein vaccines.

Groups of four female Balb/C mice will be immunized intradermally with saline or 100 μg of the following plasmid DNA constructs created in accordance with Example I: pVAX1 empty vector, pVAX-M, and pVAX-M-CD1c. An additional control group will be immunized with a construct encoding the shared leader peptide/signal sequence of CD1b fused to M in the absence of the transmembrane or cytoplasmic domains found in M-CD1c.

The product of the pVAX-M plasmid, pVAX-Sig-M, is predicted to be secreted extracellularly by transfected cells and presented by nearby professional antigen presenting cells. In addition, a vector encoding a matrix protein (M) targeted by the cytoplasmic tail of the lysosomal LAMP protein will also be used (pVAX-M-LAMP 1). Previous studies utilizing the LAMP1 targeting motif have demonstrated protection using a papillomavirus antigen.

A seventh group of animals will be immunized intradermally with 5 μg of a recombinant version of the M protein according to the above described protocol to compare DNA-based immunization platforms with traditional protein-based subunit vaccines. The data is expected show that the DNA-based immunization platform is immunologically superior to traditional protein-based subunit vaccines.

Animal immunizations will be performed three times at the base of the tail, two weeks apart. Two weeks following the final immunization, animals will be challenged with 5-10 fold the 50% lethal dose (5-10 LD₅₀) of influenza A/PR/8/34 virus (American Type Tissue Collection, Manassas Va.) and body weight is monitored daily. Animals losing 30% of their initial weight will be identified as lethally infected. The LD₅₀ value for each viral isolate being utilized will be empirically derived. Reed et al., “A simple method for estimating fifty percent endpoints” Am. J. Hyg. 27:493-497 (1938).

### EXAMPLE IV

Purification of A Recombinant Matrix Protein

This example describes a construction, expression and purification approach for a recombinant matrix protein using a bacterial expression platform.

An M cDNA plasmid created according to Example I will be ligated into the NdeI and XhoI sites of the bacterial expression vector pET22b (EMD Biosciences, San Diego Calif.). This choice of restriction sites results in the addition of the amino acid sequence LEHHHHHH (SEQ ID NO: 17) thus providing a 6×His epitope at the C terminus of the M protein. A BL-21 bacteria is transformed using the M-pET22b plasmid.

Recombinant M protein expression is induced by incubating the bacterial culture with 1 mM isopropyl-β-D-thiogalactosidase (IPTG). The expressed M protein is then purified by nickel-nitrilotriacetic acid (nickel-NTA) agarose resin affinity chromatography using recommended denaturing conditions (Qiagen, Valencia Calif.). Fractions are collected an subjected to an SDS-PAGE analysis that demonstrates the presence of a highly enriched prominent band of the appropriate size (˜28 kDa) which is recognized by the 6×His-specific reagent India Probe 6×His (Pierce, Rockford Ill.).

### EXAMPLE V

Generation Of Thapsigargin-Treated Cancer Cells

This example illustrates one embodiment for preparing thapsigargin-treated cancer cells.

4T1 mouse mammary tumor cells are available from the ATCC. This cell line demonstrates significant pro-inflammatory capacity following treatment with thapsigargin. Further, these cells are both haplotype and gender-matched with the female Balb/C mice immunizations according to Example III.

The 4T1 cells are cultured and then incubated with 2.5 μM thapsigargin for forty-eight (48) hours. Following the incubation, the cells are harvested, washed, and quantified. An ELISA-based assay is then performed to evaluate the thapsigargin-induced inflammation.

### EXAMPLE VI

CD1 Immunogen Protection Against Influenza A Protection

This example describes one approach for demonstrating that M protein immunization prevents an Influenza A infection.

A pVAX-M-CD1c construct will be used to immunize Balb/C mice according to Example III. An immunized mouse group is injected with a lethal dose of Influenza A virus. A non-immunized mouse group is also injected with a lethal dose of Influenza A virus. The data is expected to show that the immunized mouse group has a lower percentage of deaths than the non-immunized mouse group. Consequently, immunization with an M protein plasmid provides protection against lethal infection by Influenza A.

### EXAMPLE VII

LAMP Immunogen Protection Against Influenza A Protection

This example describes another approach for demonstrating that M protein immunization prevents an Influenza A infection.

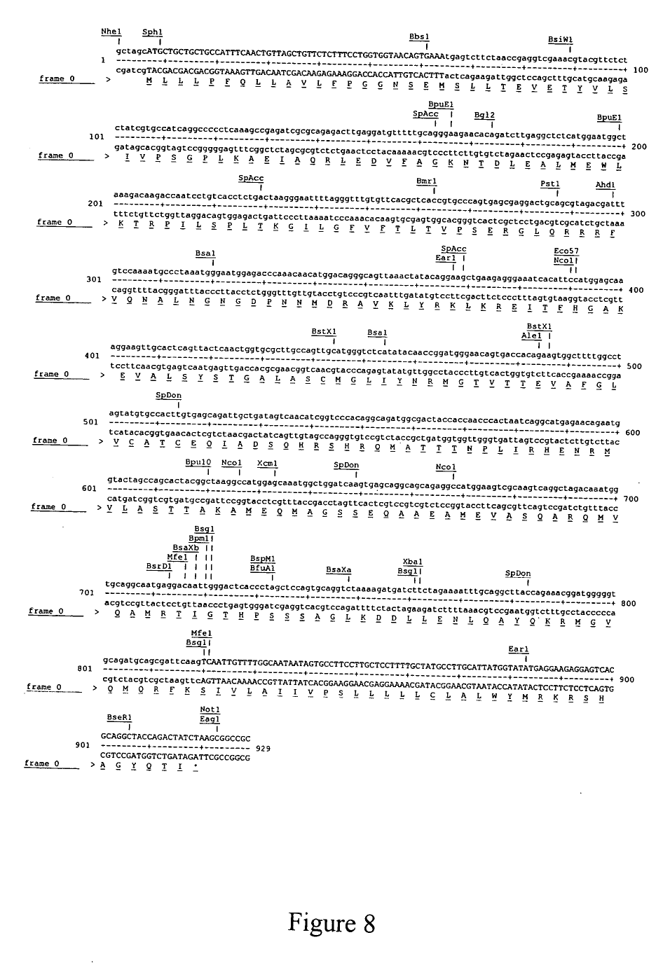

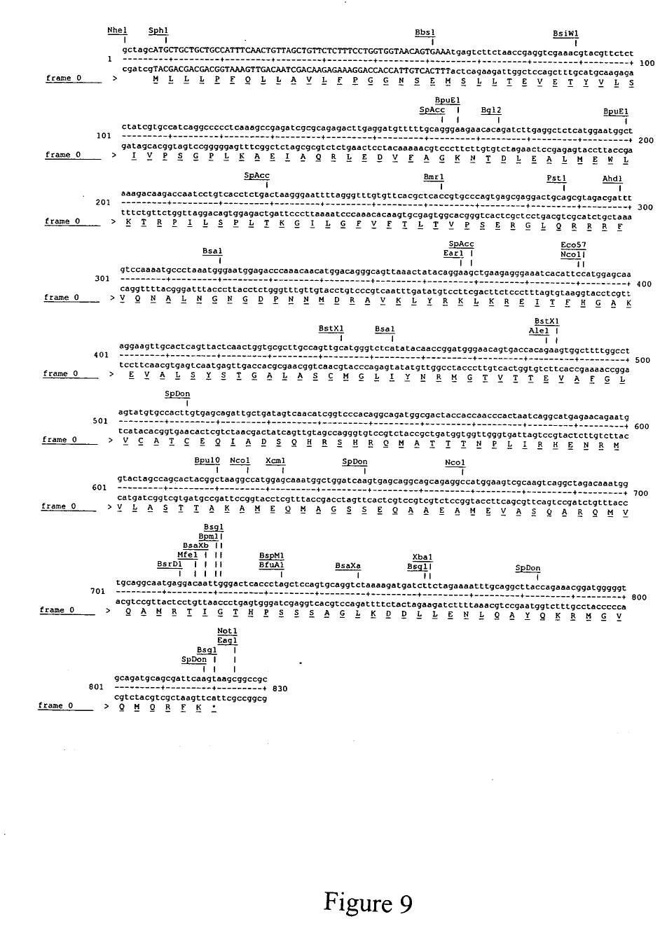

A pVAX-M-LAMP or a pVAX-Sig-M construct is used to immunize Balb/C mice according to Example III. FIG. 8 and FIG. 9, respectively. An immunized mouse group is injected with a lethal dose of Influenza A virus. A non-immunized mouse group is also injected with a lethal dose of Influenza A virus. The data is expected to show that the immunized mouse group has a lower percentage of deaths than the non-immunized mouse group. Consequently, immunization with an M protein plasmid provides protection against lethal infection by Influenza A.

### EXAMPLE VIII

Enhanced M-Protein Immunization Using Thapsigarin-Treated Cancer Cells

This example describes an approach for enhanced immunogenic response when an M protein immunogen is expressed using a thapsigarin-treated cancer cell.

Thapsigarin-treated cancer cells (TG cells) are produced according to Example V. An M protein construct produced according to either Example III or Example VI is introduced to the TG cells under conditions such that the construct is stably integrated into the TG cell's genome. The M protein immunogen is then expressed according to Example III and isolated in the presence of the TG cell extract.

The data is expected to show that TG-treated cancer cell extracts augments the immunogenic response of any M-based protein immunogen and increases the protection percentage against lethal challenge by Influenza A virus according to the protocol described in Example V and/or Example VI.

### EXAMPLE IX

In Vivo Protection

This example provides exemplary data showing that a CD1-targeted M protein could protect against an influenza infection.

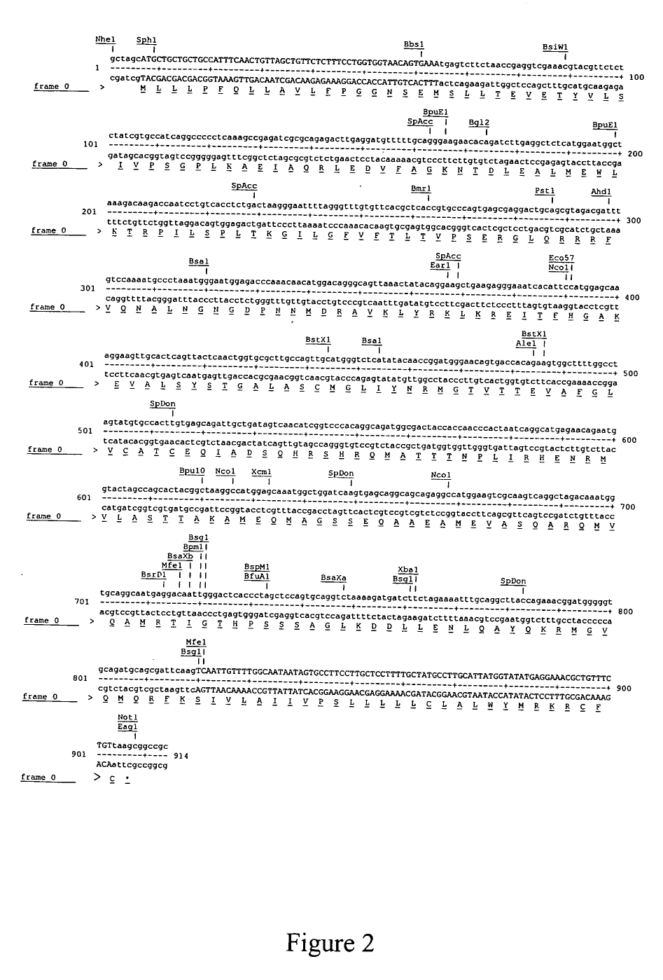

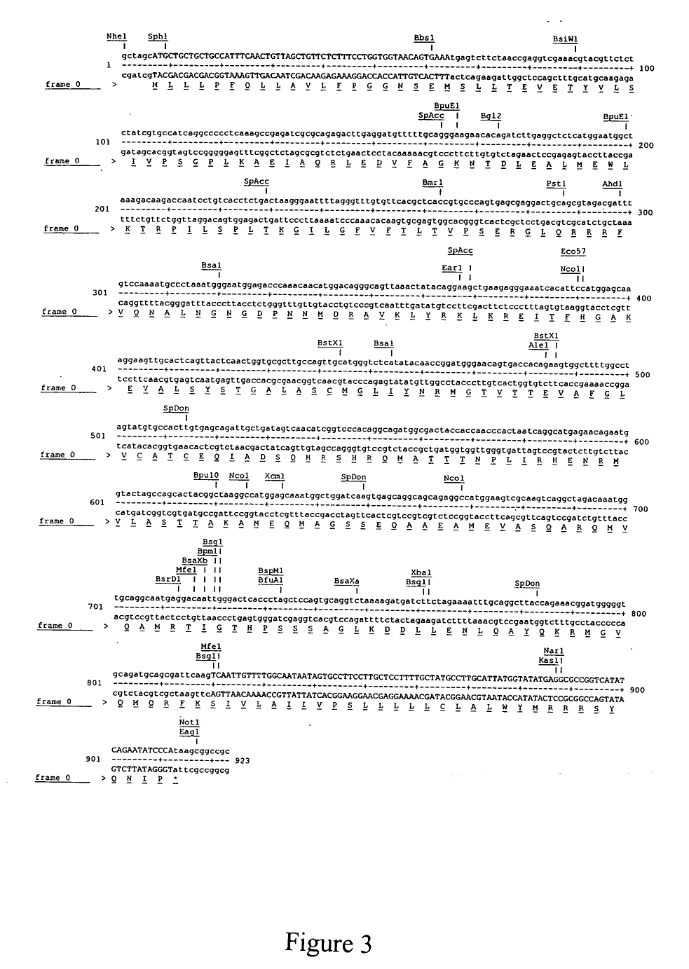

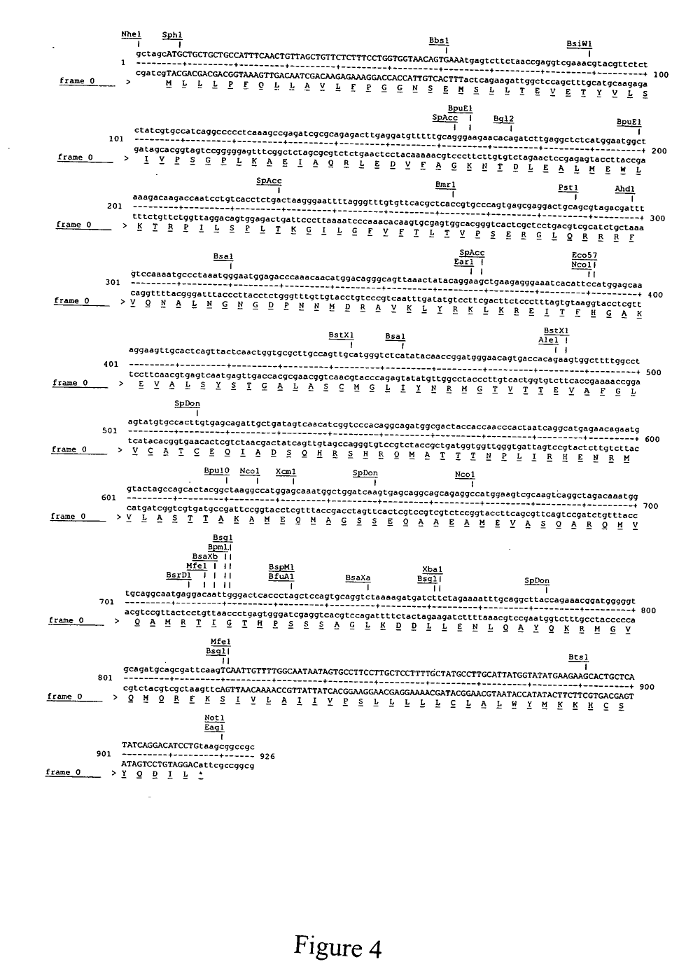

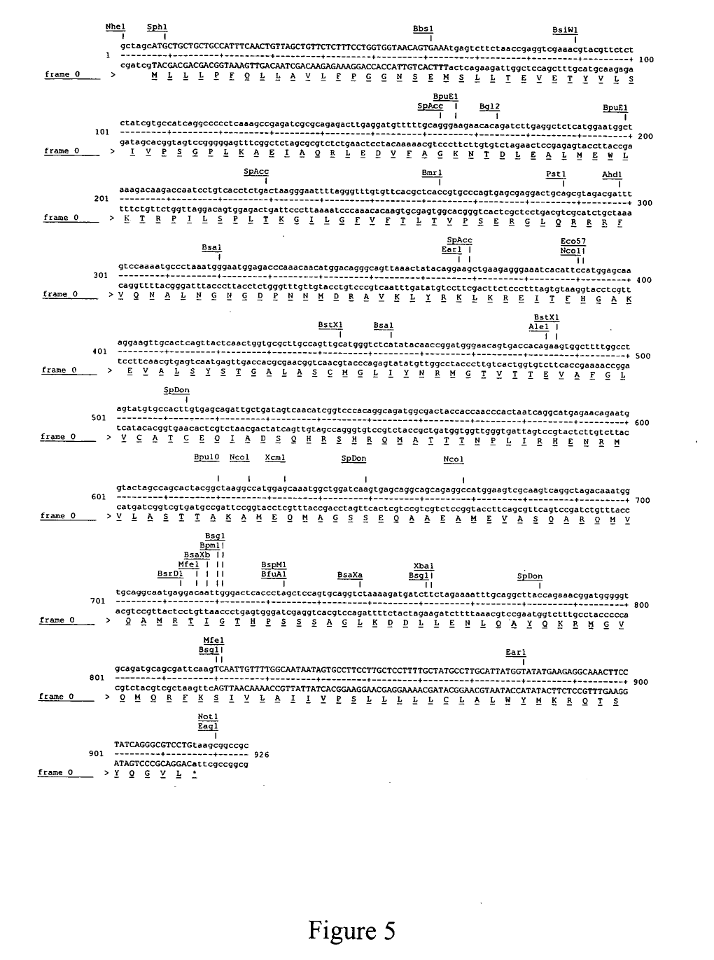

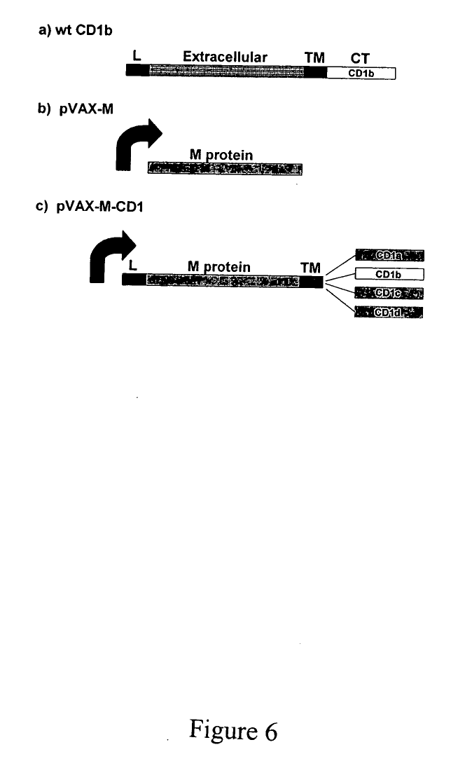

pVAX DNA expression constructs were created according to the basic protocols in Example I and Example II containing either the avian flu (H5N1) M cDNA alone or in combination with one of the following endosomal targeting sequences: i) pVAX-M-CD1a (FIG. 2); ii) pVAX-M-CD1b (FIG. 3); iii) pVAX-M-CD1c (FIG. 4), iv) pVAX-M-CD1d (FIG. 5), and v) an empty plasmid DNA. These CD1-derived constructs utilize a human CD1b leader peptide and transmembrane domain followed by an appropriate CD1 isoform-specific cytoplasmic tail endosomal targeting motif. FIG. 6.

The pVAX constructs were prepared using the Qiagen End toxin-Free DNA Giga Prep (Quailed, Valencia, Calif.) and then purified. DBA/2 (H-2^{b}) mice were immunized by an intradermal injection at the base of the tail with 100 μg of plasmid DNA and boosted after two and four weeks with repeat injections of the same plasmid DNA. Two weeks after the last immunization, each mouse was challenged with a subcutaneous injection with 250,000 genetically engineered syngeneic P815 mastocytoma cells modified to express an M protein immunogen. Tumor size development was monitored thereafter.

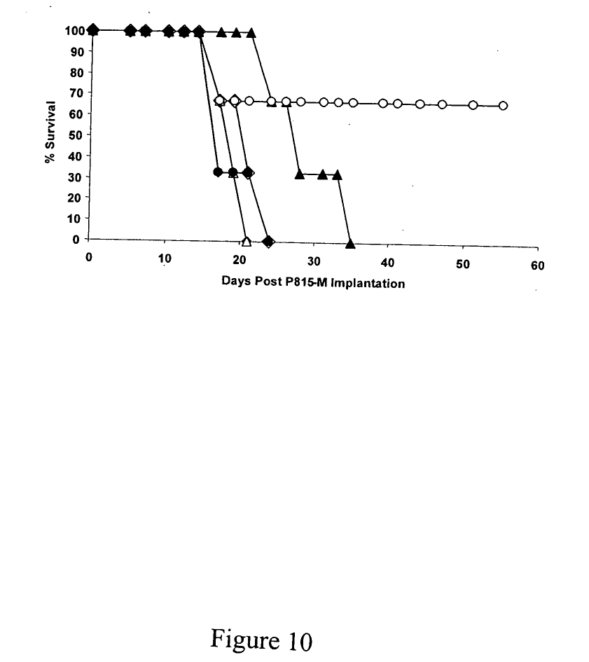

Of the plasmids tested, 2 of 3 mice immunized with an M protein targeted with a CD1c tail survived the P815 mastocytoma cell. All other groups, however, succumbed to mastocytoma cell cancer. FIG. 10. Specifically, mice immunized with an empty vector, an (H5N1) M cDNA vector, an M-CD1a vector, and an M-CD1d vector required euthanasia by day 24 following mastocytoma cell injection. The mice immunized with an M-CD1b construct demonstrated a slight improvement over the non-responsive groups, thereby indicating a lesser degree of protection than the CD1c group.

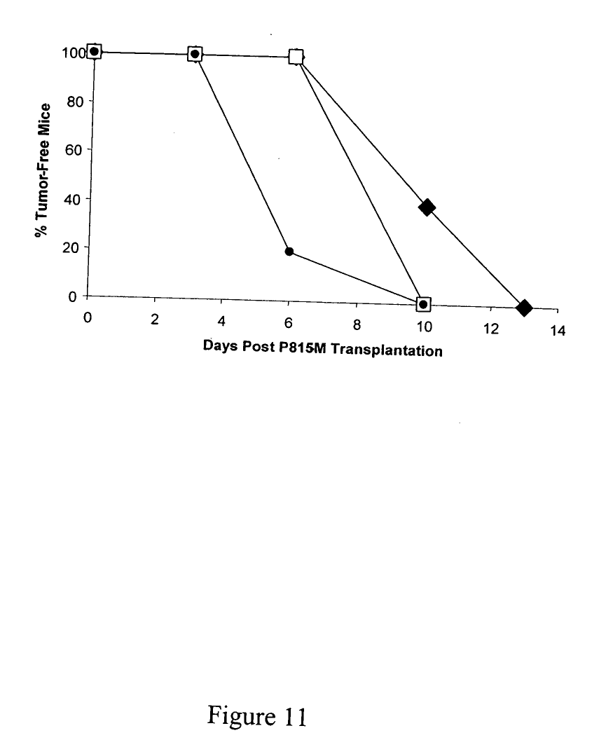

These results demonstrate that M protein vaccines can provide some protection following an avian flu injection at a concentration at least 25-fold in excess of the minimum number of cells necessary to establish a tumor. FIG. 11.

1. A vector comprising a DNA sequence encoding a fusion protein comprising a non-native viral matrix protein and one or more endosomal targeting sequences.

2. The vector of claim 1, wherein said endosomal targeting sequence is a CD1 sequence.

3. The vector of claim 1, wherein said endosomal targeting sequence is a LAMP-1 sequence.

4. The vector of claim 1, wherein said non-native viral matrix protein is greater than 90% homologous to a native Influenza A viral matrix protein.

5. The vector of claim 1, wherein said non-native viral matrix protein is greater than 95% homologous to a native Influenza A viral matrix protein

6. The vector of claim 1, wherein said non-native viral matrix protein is greater than 95% homologous to the Influenza A viral matrix proteins of at least two strains.

7. The vector of claim 1, wherein said non-native viral matrix protein is greater than 95% homologous to the matrix proteins of the H1N1, H3N2, and H5N1 strains.

8. The fusion protein encoded by the DNA sequence of the vector of claim 1.

9. A vector comprising a DNA sequence encoding a fusion protein comprising a non-native Influenza A viral matrix protein and one or more endosomal targeting sequences.

10. The vector of claim 9, wherein said endosomal targeting sequence is a CD1 sequence.

11. The vector of claim 9, wherein said endosomal targeting sequence is a LAMP-1 sequence.

12. The vector of claim 9, wherein said non-native Influenza A viral matrix protein is greater than 95% homologous to a native Influenza A viral matrix protein.

13. The vector of claim 9, wherein said non-native Influenza viral matrix protein is greater than 95% homologous to the Influenza A viral matrix proteins of at least two strains.

14. The vector of claim 9, wherein said non-native Influenza viral matrix protein is greater than 95% homologous to the matrix proteins of the H1N1, H3N2, and H5N1 strains.

15. The fusion protein encoded by the DNA sequence of the vector of claim 9.

16. A method, comprising: a) providing, a vector comprising a DNA sequence encoding a fusion protein comprising a non-native viral matrix protein and one or more endosomal targeting sequences; and a cell capable of being transfected by said vector; b) transfecting said cell with said vector so as to create a transfected cell under conditions such that said fusion protein is expressed.

17. The method of claim 16, wherein said cell capable of being transfected is cultured ex vivo.

18. The method of claim 17, wherein said transfected cell is introduced into a host.

19. A method, comprising: a) providing, a vector comprising a DNA sequence encoding a fusion protein comprising a non-native viral matrix protein and one or more endosomal targeting sequences; and a host; b) administering said vector to said host under conditions such that said fusion protein is expressed.

20. The method of claim 19, wherein said non-native viral matrix protein is greater than 90% homologous to a native Influenza A viral matrix protein.

21. The method of claim 20, wherein said host is a bird.

22. The method of claim 20, wherein said host is a human


SEQUENCE LISTING (17 sequences)

SEQ ID NO: 1 - DNA, 773 bp, Artificial Sequence
  Synthetic
gctagcatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaagtaagcggccgc

SEQ ID NO: 2 - protein, 252 aa, Artificial Sequence
  Synthetic
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFK

SEQ ID NO: 3 - DNA, 914 bp, Artificial Sequence
  Synthetic
gctagcatgctgctgctgccatttcaactgttagctgttctctttcctggtggtaacagtgaaatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaagtcaattgttttggcaataatagtgccttccttgctccttttgctatgccttgcattatggtatatgaggaaacgctgtttctgttaagcggccgc

SEQ ID NO: 4 - protein, 299 aa, Artificial Sequence
  Synthetic
MLLLPFQLLAVLFPGGNSEMSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFKSIVLAIIVPSLLLLLCLALWYMRKRCFC

SEQ ID NO: 5 - DNA, 923 bp, Artificial Sequence
  Synthetic
gctagcatgctgctgctgccatttcaactgttagctgttctctttcctggtggtaacagtgaaatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaagtcaattgttttggcaataatagtgccttccttgctccttttgctatgccttgcattatggtatatgaggcgccggtcatatcagaatatcccataagcggccgc

SEQ ID NO: 6 - protein, 302 aa, Artificial Sequence
  Synthetic
MLLLPFQLLAVLFPGGNSEMSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFKSIVLAIIVPSLLLLLCLALWYMRRRSYQNIP

SEQ ID NO: 7 - DNA, 926 bp, Artificial Sequence
  Synthetic
gctagcatgctgctgctgccatttcaactgttagctgttctctttcctggtggtaacagtgaaatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaagtcaattgttttggcaataatagtgccttccttgctccttttgctatgccttgcattatggtatatgaagaagcactgctcatatcaggacatcctgtaagcggccgc

SEQ ID NO: 8 - protein, 303 aa, Artificial Sequence
  Synthetic
MLLLPFQLLAVLFPGGNSEMSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFKSIVLAIIVPSLLLLLCLALWYMKKHCSYQDIL

SEQ ID NO: 9 - DNA, 926 bp, Artificial Sequence
  Synthetic
gctagcatgctgctgctgccatttcaactgttagctgttctctttcctggtggtaacagtgaaatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaagtcaattgttttggcaataatagtgccttccttgctccttttgctatgccttgcattatggtatatgaagaggcaaacttcctatcagggcgtcctgtaagcggccgc

SEQ ID NO: 10 - protein, 303 aa, Artificial Sequence
  Synthetic
MLLLPFQLLAVLFPGGNSEMSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFKSIVLAIIVPSLLLLLCLALWYMKRQTSYQGVL

SEQ ID NO: 11 - DNA, 797 bp, Artificial Sequence
  Synthetic
gctagcatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaaggattacaaggatgacgacgataagtaagcggccgc

SEQ ID NO: 12 - protein, 260 aa, Artificial Sequence
  Synthetic
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFKDYKDDDDK

SEQ ID NO: 13 - DNA, 929 bp, Artificial Sequence
  Synthetic
gctagcatgctgctgctgccatttcaactgttagctgttctctttcctggtggtaacagtgaaatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaagtcaattgttttggcaataatagtgccttccttgctccttttgctatgccttgcattatggtatatgaggaagaggagtcacgcaggctaccagactatctaagcggccgc

SEQ ID NO: 14 - protein, 304 aa, Artificial Sequence
  Synthetic
MLLLPFQLLAVLFPGGNSEMSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFKSIVLAIIVPSLLLLLCLALWYMRKRSHAGYQTI

SEQ ID NO: 15 - DNA, 830 bp, Artificial Sequence
  Synthetic
gctagcatgctgctgctgccatttcaactgttagctgttctctttcctggtggtaacagtgaaatgagtcttctaaccgaggtcgaaacgtacgttctctctatcgtgccatcaggccccctcaaagccgagatcgcgcagagacttgaggatgtttttgcagggaagaacacagatcttgaggctctcatggaatggctaaagacaagaccaatcctgtcacctctgactaagggaattttagggtttgtgttcacgctcaccgtgcccagtgagcgaggactgcagcgtagacgatttgtccaaaatgccctaaatgggaatggagacccaaacaacatggacagggcagttaaactatacaggaagctgaagagggaaatcacattccatggagcaaaggaagttgcactcagttactcaactggtgcgcttgccagttgcatgggtctcatatacaaccggatgggaacagtgaccacagaagtggcttttggcctagtatgtgccacttgtgagcagattgctgatagtcaacatcggtcccacaggcagatggcgactaccaccaacccactaatcaggcatgagaacagaatggtactagccagcactacggctaaggccatggagcaaatggctggatcaagtgagcaggcagcagaggccatggaagtcgcaagtcaggctagacaaatggtgcaggcaatgaggacaattgggactcaccctagctccagtgcaggtctaaaagatgatcttctagaaaatttgcaggcttaccagaaacggatgggggtgcagatgcagcgattcaagtaagcggccgc

SEQ ID NO: 16 - protein, 271 aa, Artificial Sequence
  Synthetic
MLLLPFQLLAVLFPGGNSEMSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEVASQARQMVQAMRTIGTHPSSSAGLKDDLLENLQAYQKRMGVQMQRFK

SEQ ID NO: 17 - protein, 23 aa, Homo sapiens
MDAMKRGLCCVLLLCGAVFVSPS